(19) United States
(12) Patent Application Publication
(10) Pub. No.: US 2022/0001103 A1
(43) Pub. Date: Jan. 6, 2022

(54) INTEGRATED MULTI-MEDICATION TREATMENT DELIVERY SYSTEM

(71) Applicant: University of Washington, Seattle, WA (US)

(72) Inventors: Ryan Joseph Jense, Seattle, WA (US); Martha Claire Johnson, Seattle, WA (US); Leo Li Lam, Seattle, WA (US); Thomas Andrew Bowdle, Seattle, WA (US)

(73) Assignee: University of Washington, Seattle, WA (US)

(21) Appl. No.: 17/283,318

(22) PCT Filed: Oct. 9, 2019
(86) PCT No.: PCT/US2019/055346

§ 371 Date: Apr. 7, 2021

Related U.S. Application Data
(60) Provisional application No. 62/743,394, filed on Oct. 9, 2018.

(51) Int. Cl. A61M 5/14; A61M 5/142; A61M 5/168; G16H 10/60; G16H 20/17

(52) U.S. Cl. CPC A61M 5/1407; A61M 5/142; A61M 5/16827; G16H 10/60; A61M 2005/14208; A61M 2205/3331; A61M 2205/502; A61M 2205/52; A61M 2205/18; G16H 20/17

(57) ABSTRACT

Treatment delivery apparatuses include a treatment administration device, a multi-lumen catheter reversibly couplable with the treatment administration device, and a controller. The treatment administration device has a housing cartridge-pump interfaces disposed within the housing, each cartridge-pump interface being reversibly couplable with a medication cartridge type. The treatment administration device further includes pumps disposed within the housing, each pump being in fluidic connection with at least one cartridge-pump interface. The controller enables the treatment administration device to: receive a treatment selection, determine available medications based upon medication cartridges coupled with the cartridge-pump interfaces, and administer a treatment to the multi-lumen catheter, based at least upon the treatment selection.

### CROSS-REFERENCE TO RELATED APPLICATIONS

This application claims the benefit under 35 U.S.C. § 119 to U.S. Provisional Patent Application No. 62/743,394, filed Oct. 9, 2018, which is hereby incorporated by reference in its entirety.

### BACKGROUND

Administration of intravenous (IV) medication is ubiquitous throughout the practice of modern medicine. While this mode of medication delivery has been standardized and improved over the past fifty years, there have been no major breakthroughs in recent time. Presently, IV medications are most commonly delivered by separate, individualized machines that are connected to dedicated IV lines for each medication to be given. This architecture requires multiple steps to be performed for each additional IV medication.

For example, if a patient reports to the emergency room with severe heart failure, a provider (e.g., a physician) must start multiple IV medications simultaneously to save the patient. This first requires a written order for the medications to be filled by the pharmacy and then delivered to the correct location in the hospital. The physician or nurse must then inventory each medication individually to assess general accuracy. After deciding which medication is needed first, the provider must then either manually input the medication name, concentration, and rate into the pump, or physically scan a barcode that enters the information. The provider must then fill the IV tubing with the medication to remove all the air, and then manually attach it to the pump for delivery. Next, the provider must attach this tubing to the patient and start the pump. This process must be done for each additional medication needed for treatment. An average surgery patient receives six different IV medications. These steps are tedious, time-consuming, and represent potential for error at each level of input. Importantly, for each additional medication needed, the complexity of care exponentially increases due to potential interactions. In addition, the “dead-space” resulting from the size and configuration of current tubing creates a delivery system that is slow to respond to input changes made by providers at the pump level. This can result in delayed treatment, which can adversely impacting patient outcomes.

There are five areas for improvement related to this problem: medication error, medication accountability, medication waste, medication delivery dynamics, and workspace design.

Medication errors represent the most common type of medical error in the United States. These errors range from improper dosing, incorrect type, timing, or labeling; and are reported to occur in up to 5% of hospital admissions at an estimated cost of $20 billion dollars per annum. Even in the most tightly controlled high-tech environments, such as the modern operating rooms, provider self-reporting reveals medication error rates of approximately 1 in 200 (0.5%).

Medication accountability is another vast area for patient care improvement. Currently, most medications are transcribed into the medical record via direct human input. That is, a medical provider transcribes the amount and time for any given medication into the patient chart. These transcriptions are frequently inaccurate.

Medication waste poses economic burdens on the healthcare system and environmental burdens on the world at large. Intravenous medication waste occurs for various reasons and commonly includes medication expiration, dynamic patient conditions, and medication contamination—or the potential thereof. Medication waste is estimated to range from 15-50%.

Medication delivery dynamics are vitally important when caring for critically ill patients. Such patients require multiple simultaneous medications that must be continuously adjusted to maintain normal physiologic states. For these patients, small deviations in the amount of a medication can mean life or death. Thus, medication infusion architecture should be dynamic, accurate, and highly responsive. While the current systems allow for changes of delivery rates, the inherent dead space in such systems cause an unnecessary time delay between the input (change in delivery rate setting) and output (actual delivery rate to the patient).

Operating Room Clutter results from the ever-growing number of technologies that accompany caring for a patient in the modern surgical arena. This seemingly endless growth of equipment and supplies can make it challenging for clinicians and nurses to perform their duties in a safe and efficient manner. It is often difficult for providers to care for their patients because of the physical barriers created by antiquated and redundant technologies. Thus, medication infusion architecture should be designed for its intended workplace (e.g., operating room, emergency room, or medicine floor), and should reduce clutter.

### BRIEF SUMMARY

In an aspect, the present disclosure provides treatment delivery apparatuses which include a treatment administration device, a multi-lumen catheter reversibly couplable with the treatment administration device, and a controller. The treatment administration device has a housing and a plurality of cartridge-pump interfaces disposed within the housing, each cartridge-pump interface being reversibly couplable with a medication cartridge type. The treatment administration device further includes a plurality of pumps disposed within the housing, each pump being in fluidic connection with at least one cartridge-pump interface of the plurality of cartridge-pump interfaces. The controller is operatively connected to treatment administration device and includes a processor and logic which, when executed by the processor, causes the treatment administration device to perform operations including: receiving a treatment selection via a user interface operably connected with the controller, determining a plurality of available medications based upon a plurality of medication cartridges coupled with the plurality of cartridge-pump interfaces, and administering a treatment to the multi-lumen catheter, based at least upon the treatment selection. The treatment includes at least one available medication of the plurality of available medications. For each available medication of the treatment, administering the treatment includes controlling a flow of the available medication from at least one medication cartridge of the plurality of medication cartridges to the multi-lumen catheter.

In an embodiment, the treatment administration device includes a manifold and a catheter adaptor that are in fluidic connection with the plurality of pumps.

In an embodiment, the treatment delivery apparatus includes the user interface.

In an embodiment, the treatment delivery apparatus does not include the user interface.

In an embodiment, the user interface is disposed on a remote device or the housing.

In an embodiment, the controller is disposed in the treatment administration device.

In an embodiment, the controller includes logic which, when executed by the processor, causes the treatment delivery apparatus to perform additional operations, including: receiving patient information. In an embodiment, administering the treatment is based on the patient information. In an embodiment, the patient information is received from an electronic medical record. In an embodiment, the patient information includes at least one of an allergy, an age, a weight, an ethnicity, a medical history, a medication history, a comorbidity, an ASA status, and/or a sex. In an embodiment, the controller includes logic which, when executed by the processor, causes the treatment delivery apparatus to perform additional operations, including: identifying a risk based upon the patient information and the treatment selection, displaying a warning on the user interface based upon the risk, and controlling administration of the treatment based upon the warning.

In an embodiment, the controller includes logic which, when executed by the processor, causes the treatment delivery apparatus to perform additional operations, including: identifying an interaction between at least two of the plurality of available medications, displaying a warning on the user interface based upon the interaction and the treatment selection, and controlling administration of the treatment based upon the warning.

In an embodiment, the controller includes logic which, when executed by the processor, causes the treatment delivery apparatus to perform additional operations, including: determining a safe dosage for each available medication of the treatment. In an embodiment, administering the treatment includes preventing administration of more than the safe dosage for each available medication of the treatment (such as by imposing a safe dosage limit and/or by displaying a warning on the user interface, which a user may override). In an embodiment, the controller determines the safe dosage by referencing a master drug list.

In an embodiment, administering the treatment includes, for each available medication of the treatment, determining an administered dosage and an administered time. In an embodiment, the controller includes logic which, when executed by the processor, causes the treatment delivery apparatus to perform additional operations, including: recording the administered dosage and the administered time on a memory of the controller.

In an embodiment, the controller includes logic which, when executed by the processor, causes the treatment delivery apparatus to perform additional operations, including: presenting a mode menu, receiving a mode selection from the mode menu, and presenting a different option set on the user interface for each mode selection. In an embodiment, the mode menu includes at least one of an emergency mode, a surgical floor mode, an anesthesia mode, a medicine floor mode, an intensive care unit (ICU) mode, a pediatric mode, and/or a target controlled infusion mode. In an embodiment, each of the emergency mode, the surgical floor mode, the anesthesia mode, the medicine floor mode, the intensive care unit (ICU) mode, the pediatric mode, and the target controlled infusion mode have a different option set.

In an embodiment, receiving the treatment selection includes receiving a target controlled infusion input set that includes at least one of: a target anesthetic state, a target pain state, a target memory state, or a target hemodynamic state, and administering the treatment is based upon the target controlled infusion input set. In an embodiment, the multi-lumen catheter includes a plurality of minor lumens running along a major lumen of the multi-lumen catheter. In an embodiment, administering the treatment includes, for each available medication of the treatment, controlling the flow of the available medication from at least one medication cartridge of the plurality of medication cartridges to at least one minor lumen of the multi-lumen catheter. In an embodiment, when the multi-lumen catheter is reversibly coupled with the treatment administration device, each minor lumen of the multi-lumen catheter is fluidically coupled with at least one pump. In an embodiment, each minor lumen opens into the major lumen at a location adjacent to a downstream end of the multi-lumen catheter. In an embodiment, each minor lumen opens into the major lumen at about 0.5 cm to about 5.0 cm from the downstream end of the multi-lumen catheter. In an embodiment, wherein each minor lumen radially surrounds the major lumen. In an embodiment, each minor lumen is disposed in a common plane that runs along the major lumen. In an embodiment, the major lumen has a largest cross-sectional dimension of about 1 mm to about 5 mm, and no minor lumen has a largest cross-sectional dimension that equals or exceeds the largest cross-sectional dimension of the major lumen. In an embodiment, the major lumen and each minor lumen has a circular cross-sectional shape. In an embodiment, the multi-lumen catheter includes an emergency medication port located proximal to a downstream end of the multi-lumen catheter and providing access to the major lumen. In an embodiment, the multi-lumen catheter is disposable, configured for single patient use, and is configured to prevent backflow into the treatment administration device.

In an embodiment, each cartridge-pump interface is configured to read digital information from a medication cartridge coupled thereto. In an embodiment, the digital information includes at least one information type selected from: a medication type, a medication amount, a medication concentration, a medication cartridge type, a time remaining to cartridge change, and/or an expiration date. In an embodiment, determining the plurality of available medications is based upon the digital information read by at least one cartridge-pump interface of the plurality of cartridge-pump interfaces.

In an embodiment, the plurality of cartridge-pump interfaces is arranged in a planar array proximal to an outer panel of the housing.

In an embodiment, the treatment includes fewer than all available medications of the plurality of available medications.

In an embodiment, the plurality of available medications includes a saline solution, and the treatment includes the saline solution.

In an embodiment, the treatment delivery apparatus includes a climate control apparatus configured regulate a climate within the housing.

In an embodiment, the treatment delivery apparatus includes the plurality of medication cartridges.

In another aspect, the present disclosure provides a treatment administration device that includes a housing, a plurality of cartridge-pump interfaces, and a plurality of pumps. The plurality of cartridge-pump interfaces is disposed within the housing, and each cartridge-pump interface is reversibly couplable with a medication cartridge type. The plurality of pumps is disposed within the housing, and each pump is in fluidic connection with at least one cartridge-pump interface of the plurality of cartridge-pump interfaces. The treatment administration device is configured to fluidically connect with a multi-lumen catheter.

In another aspect, the present disclosure provides treatment delivery systems which include a treatment administration device, a multi-lumen catheter reversibly couplable with the treatment administration device, and a controller. In an embodiment, the treatment delivery system includes a plurality of medication cartridges and/or a user interface.

### BRIEF DESCRIPTION OF THE SEVERAL VIEWS OF THE DRAWINGS

The foregoing aspects and many of the attendant advantages of the present disclosure will become more readily appreciated as the same become better understood by reference to the following detailed description, when taken in conjunction with the accompanying drawings, wherein:

To easily identify the discussion of any particular element or act, the most significant digit or digits in a reference number refer to the figure number in which that element is first introduced.

FIG. 1 shows a treatment delivery apparatus in accordance with one embodiment of the present disclosure;

FIG. 2 shows an aspect of a treatment administration device of a treatment delivery apparatus, in accordance with one embodiment of the present disclosure;

FIG. 3 shows another aspect of the treatment administration device of FIG. 2;

FIG. 4A and FIG. 4B show aspects of a manifold of a treatment administration device, in accordance with one embodiment of the present disclosure;

FIG. 5A and FIG. 5B show aspects of a catheter adaptor of a treatment administration device, in accordance with one embodiment of the present disclosure;

FIG. 6A and FIG. 6B show aspects of a multi-lumen catheter of a treatment delivery apparatus, in accordance with one embodiment of the present disclosure;

FIG. 7 shows aspects of a multi-lumen catheter of a treatment delivery apparatus, in accordance with another embodiment of the present disclosure;

FIG. 8 shows a user interface of a treatment delivery apparatus, in accordance with one embodiment of the present disclosure; and

FIG. 9 shows a schematic representation of a treatment delivery apparatus, in accordance with one embodiment of the present disclosure.

### DETAILED DESCRIPTION

The present disclosure provides treatment delivery apparatuses and systems configured to deliver treatments to patients from an “inventory” or “pharmacy” of available medications. The treatment delivery apparatus utilizes a number of novel elements that collectively reduce treatment errors, improve treatment accountability, reduce treatment waste, improve treatment delivery dynamics, and improve workplace design. As used herein, a “treatment” includes one or more substances (e.g., liquid medications, liquid formulations, liquid solutions, etc.). Thus, “treatment” includes single-substance treatments and multi-substance treatments. Although the present disclosure generally describes treatments in the context of medications, the inventors contemplate that the inventive systems are suitable for treatments that do not include medications (e.g., treatment including cosmetic substances).

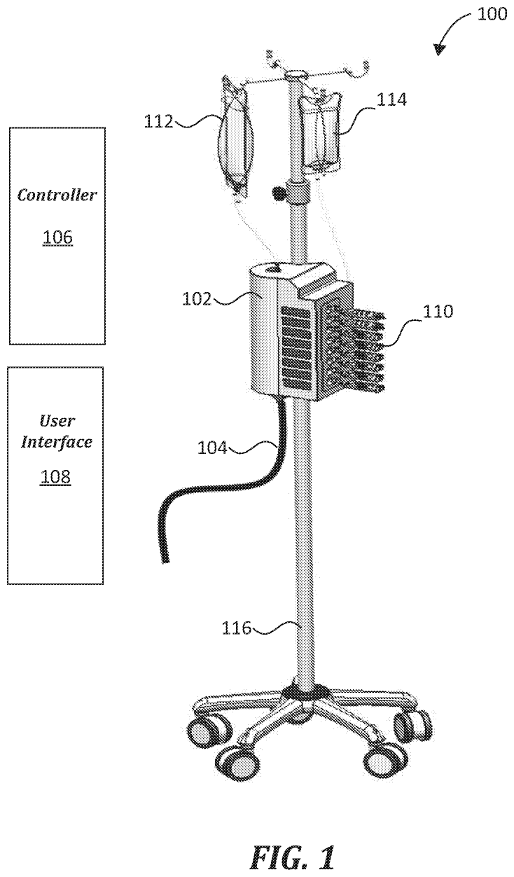

FIG. 1 shows a representative treatment delivery apparatus 100, in accordance with one non-limiting embodiment of the present disclosure. The treatment delivery apparatus 100 includes three main sub-systems that cooperate to administer a plurality of treatments: a treatment administration device 102, a multi-lumen catheter 104, and a controller 106. Each of these sub-systems is introduced with respect to FIG. 1 and described in detail with respect to the other FIGURES. To administer the treatments, the treatment delivery apparatus 100 includes a plurality of independent fluidic flow paths, each of which begins in the treatment administration device 102 and leads into the multi-lumen catheter 104. Each independent fluidic flow path remains isolated from the other fluidic flow paths (i.e., does not mix with another fluidic flow path) through the treatment administration device 102 and through at least part of the multi-lumen catheter 104. At least a portion of each fluidic flow path is sterile or sterilizable.

The treatment administration device 102 is a device that is generally configured to support one or more medication cartridges 110, and to pump medication from one or more of the medication cartridges 110 to the multi-lumen catheter 104. The medication(s) pumped by the treatment administration device 102 to the multi-lumen catheter 104 make up the treatment. The treatment administration device 102 includes an outer housing, a plurality of cartridge-pump interfaces disposed within the housing, a plurality of pumps disposed within the housing, and a catheter adaptor that is fluidically connected to the pumps. The treatment administration device 102 includes a plurality of independent fluidic flow paths, each of which begins with one of the cartridge-pump interfaces, passes through one of the pumps, and passes through the catheter adaptor. Each fluidic flow path of the treatment administration device 102 corresponds to an independent fluidic flow path of the multi-lumen catheter 104.

The multi-lumen catheter 104 is generally configured to transport one or more medications (and thus, the treatment) from the treatment administration device 102 to a patient. The multi-lumen catheter 104 is fluidically connectable to the treatment administration device 102 via the catheter adaptor. The multi-lumen catheter 104 is configured in such a manner to improve delivery dynamics and workplace design.

The controller 106 is communicatively connectable to treatment administration device 102, and is generally configured to:
receive a treatment selection via a user interface 108 that is operably connected with the controller 106;
determine one or more available medications (i.e., an “inventory” or “pharmacy” of available medications) based upon the medication cartridges 110 and medication bags (e.g., saline bag 112 and IV bag 114) coupled with the treatment administration device 102; and
administer a treatment to the multi-lumen catheter 104, based at least upon the treatment selection. The treatment includes at least one available medication from the “inventory” or “pharmacy.” For each available medication of the treatment, the controller 106 controls a flow of the available medication from at least one of the medication cartridges 110 to the multi-lumen catheter 104.

The inventors contemplate many variations and features of the treatment delivery apparatus 100. For example, in some embodiments, the administered treatment is determined by a user (e.g., a physician). In some embodiments, the administered treatment is determined at least partially by the controller 106. In some embodiments, a portion of each fluidic flow path is replaceable and/or disposable. In some embodiments, the treatment delivery apparatus 100 includes a “flush” or “prep” mode that flushes one or more of the fluidic channels with a saline solution or another inert solution (such as provided by one or more of the medication cartridges 110 and/or from a separate sterile cartridge or reservoir). In some embodiments, the treatment delivery apparatus 100 may be configured to flush fluids to an external receptacle (e.g., a drain pan). In some embodiments, the “flush” or “prep” mode is an automatic function that occurs each time a portion of the fluidic flow path is replaced or removed. In some embodiments, the treatment administration device 102 houses at least part of the controller 106. In some embodiments, the treatment delivery apparatus 100 includes one or more of the medication cartridges 110. In some embodiments, the treatment delivery apparatus 100 includes an IV rack 116. In some embodiments, the treatment delivery apparatus 100 includes the saline bag 112 and/or one or more of the IV bag 114. Additional variations and features are described below.

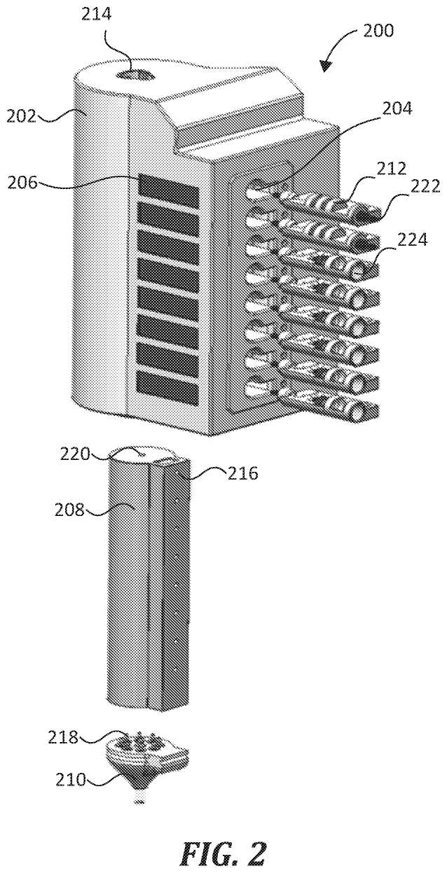

FIG. 2 shows a representative treatment administration device 200, shown partially exploded. The treatment administration device 200 is a device that is generally configured to support one or more medication cartridges, medication bags, and potentially other medication vessels, and to pump medication from one or more of the medication cartridges to a multi-lumen catheter via a plurality of independent fluidic flow paths.

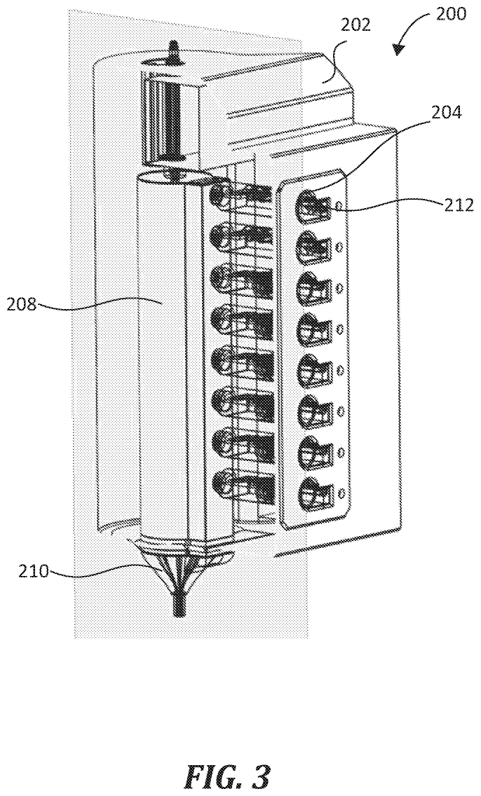

The treatment administration device 200 includes a housing 202, a plurality of cartridge-pump interfaces 204, a plurality of pumps 206, a manifold 208, and a catheter adaptor 210 (hereinafter “adaptor”). The treatment administration device 200 includes a plurality of independent fluidic flow paths, each extending from one of the cartridge-pump interfaces 204, through one of the pumps 206, through the manifold 208, and through the adaptor 210. In an assembled state (as shown in FIG. 3), the manifold 208 is received within an internal cavity of the treatment administration device 200 (not shown in FIG. 2).

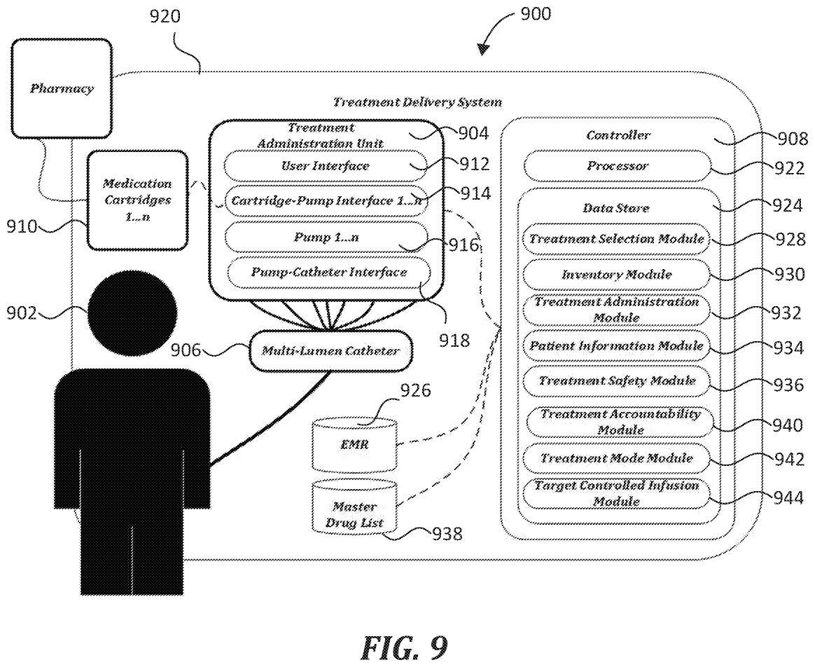

The housing 202 provides a secure and sanitary enclosure that is configured to support the cartridge-pump interfaces 204, the pumps 206, the manifold 208, and the adaptor 210. Additionally, the housing 202 is configured to support (via the cartridge-pump interfaces 204) a plurality of medication cartridges 212. In some embodiments, a controller (such as the controller 106 of FIG. 1 and the controller 908 of FIG. 9) is at least partially disposed in the housing 202. In some embodiments, a user interface, e.g., a touchscreen, is at least partially disposed in or on the housing 202. In some embodiments, the housing 202 is formed at least partially from one or more medical grade plastics, e.g., [list appropriate materials].

The cartridge-pump interfaces 204 are each configured to reversibly and fluidically couple one of the medication cartridges 212 to one of the pumps 206, such that each of the pumps 206 can pump medication from one of the medication cartridges 212. In some embodiments, at least one of the cartridge-pump interfaces 204 includes a fluid coupling that is configured to receive one of the medication cartridges 212, such as a locking or sliding Luer lock-type fluid coupling. In some embodiments, at least one of the cartridge-pump interfaces 204 utilizes a reversible latch to retain the one of the medication cartridges 212 therein. Representative latches include twist locking latches, single-touch push-push latches, latches having a cradle that latchingly receives one of the medication cartridges 212, magnetic latches, latches utilizing fingers and/or engagement members configured to releasably engage one of the medication cartridges 212, and the like. In some embodiments, the treatment administration device 200 includes cartridge-pump interfaces 204 having more than one type of fluid coupling, latch, and/or other mechanism.

The cartridge-pump interfaces 204 are arranged to provide convenient access for a user. In the non-limiting embodiment of FIG. 2, the cartridge-pump interfaces 204 are arranged in a planar array proximal to an outer panel of the housing 202 (e.g., a rear panel or side panel). In FIG. 2, the cartridge-pump interfaces 204 are vertically-arranged. By “vertically arranged,” the present description refers to orientations where at least one of the cartridge-pump interfaces 204 is located at least partially gravitationally above and at least partially over another of the cartridge-pump interfaces 204 when the treatment administration device 200 is positioned for operation. In some embodiments, the cartridge-pump interfaces 204 are horizontally arranged in a side wall. By “horizontally arranged,” the present description refers to orientations where at least one of the cartridge-pump interfaces 204 is located next to (and not over) another of the cartridge-pump interfaces 204 when the treatment administration device 200 is positioned for operation. In some embodiments, the cartridge-pump interfaces 204 are arranged in a geometrically array (e.g., a 3×3 array, a 4×4 array, or a 5×5 array). The embodiment shown in FIG. 2 includes eight cartridge-pump interfaces 204. Some embodiments may include fewer or additional cartridge-pump interfaces, e.g., 5, 10, 20, 30, 40, 50, etc. In some embodiments, the cartridge-pump interfaces include one or more sensors, radiofrequency identification (RFID) devices, and/or near field communication (NFC) devices, e.g., that are configured recognize digitally identifiable information from one or more medication cartridges coupled thereto.

The pumps 206 are each precision pumps configured to deliver precise dosages of liquid medication. In some embodiments, the pumps 206 include one or more peristaltic pumps, which are suitable to draw relatively large volumes of medications (e.g., about 1 ml to about 500 ml) from the medication cartridges 212. In some embodiments, the pumps 206 include one or more screw/syringe type pumps (which include a piston driven by a stepper motor, for example), which are suitable to deliver relatively small dosages from the medication cartridges 212, e.g., about 0.01 ml to about 5 ml. In some embodiments, the pumps 206 include one or more centrifugal pumps. The pumps 206 are electrically connected to a controller, as described below, and to a power source (e.g., a 120V AC power source). In some embodiments, the treatment administration device 200 includes more than one type of pump.

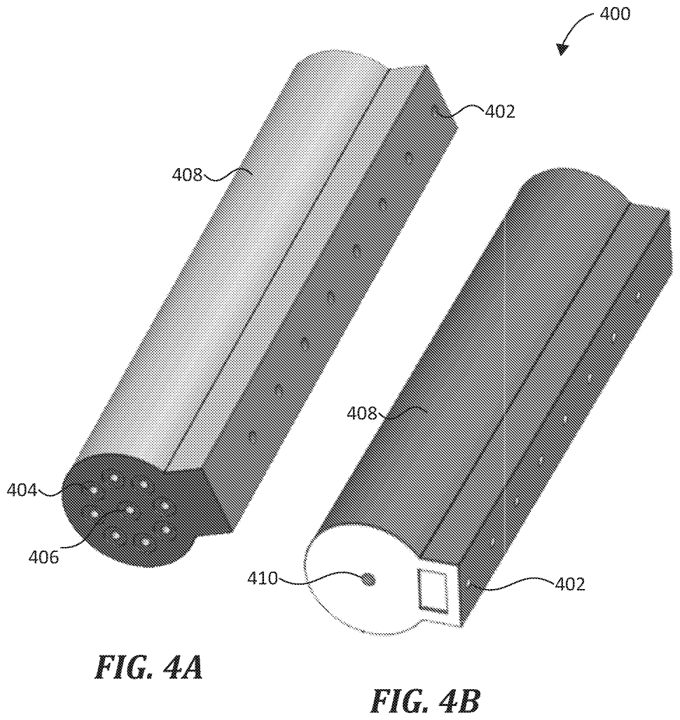

The manifold 208 includes a plurality of independent fluidic flow paths, each of which connects at an upstream end with one of the pumps 206 and at a downstream end to the adaptor 210. In the assembled state, the adaptor 210 couples with the downstream end of the manifold 208. The manifold 208 includes manifold medication entry ports 216 that are each configured to fluidically connect to the downstream/outlet of one of the pumps 206. The manifold 208 of FIG. 2 also includes a manifold auxiliary medication entry port 220, which is configured to receive a gravity-fed or pump-regulated (such as with a peristaltic pump) medication supply (such as a from the saline bag 112 or IV bag 114 of FIG. 1) via the auxiliary medication interface 214. At the downstream end, the manifold 208 includes manifold medication exit ports (shown in FIG. 4A) that are each configured to fluidically connect to one of the adaptor medication entry ports 218 of the adaptor 210. The manifold 208 has a key-shaped cross section in FIG. 2 to facilitate alignment during assembly with the housing 202, such that each of the manifold medication entry ports 216 fluidically connects to one of the pumps 206. In some embodiments, the manifold 208 may have a different cross-sectional shape (e.g., rectangular, triangular, oval-shaped, etc.). Another representative manifold is described below with respect to FIG. 4A and FIG. 4B.

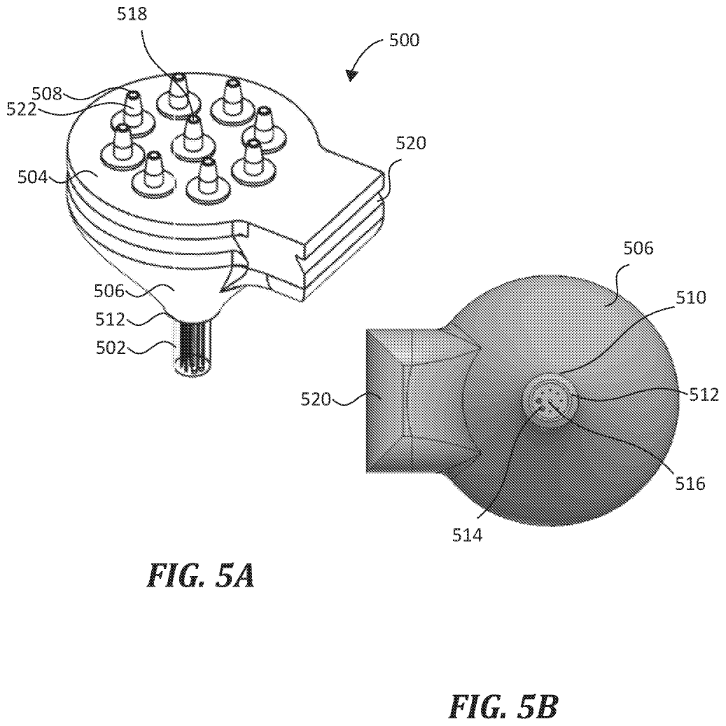

The adaptor 210 is configured to consolidate at least some of the independent fluidic flow paths from the treatment administration device 200 into a smaller cross-sectional grouping that corresponds to a multi-lumen catheter. The adaptor 210 includes a plurality of fluidic flow paths, each of which connects with a fluidic flow path of the manifold 208. The fluidic flow paths remain independent (i.e., isolated) within the adaptor 210. The adaptor 210 has an upstream portion with a first cross sectional area, and a downstream portion with a second, smaller, cross sectional area. The downstream portion of the adaptor 210 is configured to fluidically couple with a multi-lumen catheter. Another representative adaptor is described below with respect to FIG. 5A and FIG. 5B.

The medication cartridges 212 are each configured to provide a medication (e.g., a liquid medication) to one of the pumps 206. FIG. 2 shows representative and non-limiting medication cartridges 212. The medication cartridges 212 of FIG. 2 include a first type that is configured to fluidically connect with a medication bag via a fitting 222, and a second type that is configured to receive a medication vial within a vial carriage 224. In other embodiments, the treatment administration device 200 is configured to interface with medication cartridges having different features. In some embodiments, at least one of the medication cartridges 212 utilizes an ink-jet type mechanism or other electromechanical mechanism to dispense liquid. In some embodiments, at least one of the medication cartridges 212 utilizes a syringe-like design. In some embodiments, each of the medication cartridges 212 contains digitally identifiable information (which may be read by the treatment administration device 200). In some embodiments, the digitally identifiable information may be represented as a 2-D bar code, a 2-D quick response (“QR”) code, a 3-D code, a pattern of time-correlated flashing visible light or other electromagnetic signals, or other similar format. In some embodiments, digitally identifiable information associated with the medication cartridges 212 includes a medication type, medication amount, a medication concentration, a medication cartridge “type,” and a time remaining to cartridge change (an expiration date). The medication cartridges 212 may have any standardized volume, e.g., 1 ml, 5 ml, 50 ml, 100 ml, 500 ml, etc. In some embodiments, the medication cartridges 212 may include one or more optional sensors, radiofrequency identification (RFID) devices, and/or near field communication (NFC) devices to communicate the foregoing digital information.

In some embodiments, each fluidic flow path of the treatment administration device 200 is fluidically couplable to a saline source or other inert solution, e.g., at each of the cartridge-pump interfaces 204. This way, the saline source (e.g., saline bag 112 of FIG. 1) can “flush” or “prep” one or more of the fluidic channels in certain circumstances. In some embodiments, the saline source flushes a fluidic channel of the treatment administration device 200 each time a portion of the fluidic flow path is replaced or removed, including the manifold 208, the adaptor 210, and/or a multi-lumen catheter. In some embodiments, the treatment administration device 200 includes a climate control apparatus configured regulate a climate within the housing 202.

FIG. 3 shows a partial section view of the treatment administration device 200 of FIG. 2 in an assembled state. As shown, the manifold 208 is received within an internal cavity of the housing 202, and the adaptor 210 is fluidically coupled with the downstream end of the manifold 208. Each of the medication cartridges 212 is received within one of the cartridge-pump interfaces 204 such that it fluidically communicates with one of the pumps 206 (not shown in FIG. 3), with the manifold 208, and with the adaptor 210. Thus, the treatment administration device 200 includes a plurality of independent fluidic flow paths, each leading to the adaptor 210 from where one of the medication cartridges 212 couples with one of the cartridge-pump interfaces 204.

FIG. 4A and FIG. 4B show two views of a representative manifold 400 that is similar to the manifold 208 of FIG. 2. Manifold 400 includes a manifold body 408 with a plurality of fluidic flow paths formed therein. Manifold 400 has a key-shaped cross-sectional shape, to ensure that manifold 400 has the correct orientation when inserted into a treatment administration device (e.g., the treatment administration device 200 of FIG. 2). The manifold body 408 includes a plurality of manifold medication entry ports 402 and a plurality of manifold medication exit ports 404 (eight each in the non-limiting embodiment shown). Each of the manifold medication entry ports 402 fluidically connects via an isolated fluidic flow path to one of the manifold medication exit ports 404. Manifold 400 also includes a manifold auxiliary medication entry port 410, which fluidically connects via an isolated fluidic flow path to a manifold auxiliary medication exit port 406. Thus, the manifold 400 includes nine isolated fluidic flow paths. The illustrated manifold 400 is substantially formed in a single piece (e.g., such as through additive manufacturing). In some embodiments, manifold 400 is formed from a plurality of pieces that assemble together.

In some embodiments, the manifold 400 has a different number of fluidic flow paths and/or a different arrangement of fluidic flow paths. In some embodiments, the manifold 400 is formed substantially from a medical grade polymer. In some embodiments, the manifold 400 is disposable and designed for single-patient use. Thus, in some methods of use, the manifold 400 is disposed of after use in connection with a single patient. In some embodiments, the manifold 400 is part of a kit of disposable components along with a treatment administration device, a multi-lumen catheter adaptor and/or a multi-lumen catheter.

FIG. 5A and FIG. 5B show two views of a representative catheter adaptor 500 (hereinafter “adaptor”), which is similar to the adaptor 210 of FIG. 2. The adaptor 500 is configured for reversible coupling to a treatment administration device (e.g., treatment administration device 200 of FIG. 2) via a downstream portion of a manifold (e.g., manifold 400). The adaptor 500 is also configured for reversible coupling to a multi-lumen catheter.

The adaptor 500 is configured to consolidate all independent fluidic flow paths from the treatment administration device into a smaller cross-sectional grouping that corresponds to a multi-lumen catheter. To facilitate understanding, the adaptor 500 is shown coupled to a section of a multi-lumen catheter 502. The adaptor 500 has an upstream portion 504 with a first cross sectional area and a plurality of medication entry ports 508, and a downstream portion 506 with a plurality of medication exit ports 510 and a second cross sectional area that is smaller than the upstream portion 504. The downstream portion 506 has a recessed female portion 512 that is sized to receive the multi-lumen catheter 502.

The medication entry ports 508 include radial outer medication entry ports (e.g., where 508 points) and a central auxiliary entry port 518. The radial outer medication entry ports 508 are each configured to receive medication from a single manifold medication exit port (such as one of the manifold medication exit ports 404 of FIG. 4A). The central auxiliary entry port 518 is configured to receive medication from the manifold auxiliary medication exit port (e.g., manifold auxiliary medication exit port 406 of FIG. 4A). Other embodiments may have a different number or arrangement of entry ports. Each of the medication entry ports 508 and the auxiliary entry port 518 is fitted with an optional check valve 522 to prevent backflow into the manifold and/or treatment administration device.

The medication exit ports 510 include radial outer medication entry ports (e.g., where 510 points) and a central auxiliary exit port 516. The radial outer medication exit ports 510 are each fluidically connected to one of the medication entry ports 508. The auxiliary exit port 516 is fluidically connected to the auxiliary entry port 518. Other embodiments may have a different number or arrangement of exit ports. In the illustrated embodiment, the medication exit ports 510 have different diameters to enable different treatment rates. For example, the medication exit port 510 has a first diameter, and the second medication exit port 514 has a second, larger diameter. The auxiliary exit port 516 may have a diameter that is the same or different from other of the medication exit ports 510. In some embodiments, at least one of the fluidic flow paths of the adaptor may have a different diameter than at least one other of the fluidic flow paths.

In the non-limiting embodiment shown, the recessed female portion 512 has a diameter and depth that is configured to securely receive the multi-lumen catheter 502 with a friction fit. In some embodiments, the female portion 512 has a raised portion that corresponds to a detent of the multi-lumen catheter 502, which together ensure that the multi-lumen catheter 502 has the correct alignment with the adaptor 500 such that the fluidic channels of the adaptor 500 fluidically connect to the correct fluidic channels of the multi-lumen catheter 502. In some embodiments, a different mechanism may be utilized to couple and align the adaptor to the multi-lumen catheter. As one example, the adaptor may have a male portion at the downstream portion that is configured to be received by a female portion of the multi-lumen catheter. As another example, the adaptor may have a recessed female portion with a non-circular cross-sectional shape that corresponds with a non-circular cross-sectional shape of the multi-lumen catheter, such that the multi-lumen catheter can be inserted into the adaptor in the correct orientation.

To facilitate correct alignment with the treatment administration device (including the manifold), the adaptor 500 of the illustrated embodiment includes an optional alignment portion 520 that extends away from the upstream portion 504. The alignment portion 520 gives the adaptor 500 a key-shaped cross-sectional shape that corresponds to the cross-sectional shape of the manifold (e.g., the manifold 400 of FIG. 4A and FIG. 4B). In some embodiments, the adaptor and the manifold may have corresponding cross-sectional shapes that are not key-shaped. The alignment portion 520 incudes a detent that enables secure coupling, e.g., in connection with a latch on the treatment administration device. In other embodiments, the adaptor may have a latch or other securing mechanism to enable secure coupling with the treatment administration device.

In some embodiments, the adaptor 500 is formed substantially from a medical grade polymer. In some embodiments, the adaptor 500 is disposable and designed for single-patient use. Thus, in some methods of use, the adaptor 500 is disposed of after use in connection with a single patient. In some embodiments, the adaptor 500 is part of a kit of disposable components along with a manifold and/or a multi-lumen catheter.

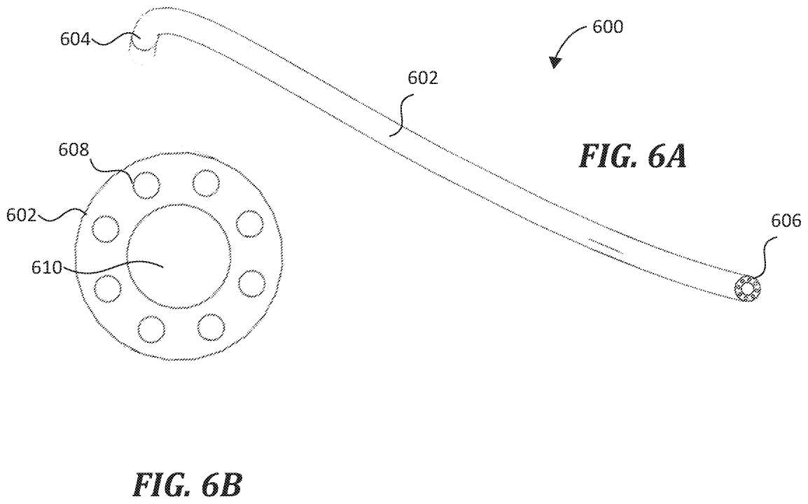

FIG. 6A shows a representative multi-lumen catheter 600 formed in accordance with an embodiment of the present disclosure. FIG. 6B shows a cross section of the multi-lumen catheter 600. The multi-lumen catheter 600 has a tubing body 602 with an upstream end 604 and a downstream end 606. The tubing body 602 may be of any convenient length (e.g., about 2 feet to about 10 feet). The upstream end 604 is configured to couple with a treatment administration device. For example, the upstream end 604 of the illustrated embodiment is configured to be received by an adaptor (such as by the recessed female portion 512 of the adaptor 500 of FIG. 5A and FIG. 5B). The downstream end 606 is configured to couple with a patient's IV (i.e., a hypodermic needle), with a reservoir, and/or with one or more hardware elements (e.g., a connector, a flow regulator, or a Luer lock) located upstream of the patient's IV.

The tubing body 602 includes a plurality of minor lumens 608 formed integrally therein that run along a major lumen 610, which is also integrally formed with the tubing body 602. The minor lumens 608 and the major lumen 610 may each run substantially along the length of the multi-lumen catheter 600. Each of the minor lumens 608 is configured to fluidically connect with one of the independent fluidic channels of the treatment administration device, and to maintain isolation of that fluidic channel along at least part of the length of the multi-lumen catheter 600. Thus, each of the minor lumens 608 is configured to fluidically connect to a pump and to a cartridge-pump interface of the treatment administration device.

The minor lumens 608 are each configured to carry a liquid medication. The major lumen 610 is configured to carry at least one liquid medication. In some embodiments, the major lumen 610 is configured to carry saline or an inert substance from the treatment administration device (for example, which may be provided via the auxiliary medication interface 214 of FIG. 2). As another example, the major lumen 610 may be configured to carry at least one medication that is provided to it by at least one of the minor lumens 608. Accordingly, in some embodiments, at least one of the minor lumens 608 opens into the major lumen 610 at a location that is adjacent to the downstream end 606, for example about 0.5 cm to about 5.0 cm from the downstream end 606. By opening into the major lumen 610 at a location adjacent to the downstream end 606, the multi-lumen catheter 600 reduces dead space. In such embodiments, the multi-lumen catheter 600 is configured such that medications carried by the minor lumens 608 mix in the major lumen 610. In such embodiments, the major lumen 610 may be configured to fluidically couple to a patient's IV (i.e., a hypodermic needle), with a reservoir, and/or with one or more hardware elements (e.g., a connector, a flow regulator) located upstream of the patient's IV.

In some embodiments, at least one of the minor lumens 608 (e.g., all minor lumens 608) opens into a reservoir adjacent to where the one or more minor lumens 608 opens into the major lumen 610. In some embodiments, the reservoir is formed integrally with the multi-lumen catheter and is part of the multi-lumen catheter. The reservoir may contain a one-way valve (e.g., a silicone diaphragm, a bi-leaflet valve, or tri-leaflet valve based on a venous/aortic valve design). The reservoir is configured to fluidically couple with a patient's IV via a Luer lock or other fitting. In some embodiments, the multi-lumen catheter includes an optional emergency medication port located adjacent to the downstream end 606 and providing access to the major lumen 610. The emergency medication port allows a user to quickly administer treatment, e.g., in an emergency.

Each of the minor lumens 608 and the major lumen 610 may be sized and shaped to improve delivery dynamics. In some embodiments, the major lumen 610 has a largest cross-sectional dimension of about 1 mm to about 5 mm, and none of the minor lumens 608 has a largest cross-sectional dimension that equals or exceeds the largest cross sectional dimension of the major lumen 610. In some embodiments, the relatively small cross-sectional area of the minor lumens 608 effectively prevents backflow of medication into the treatment administration device, thereby improving safety. In some embodiments, at least one of the minor lumens has a different largest cross-sectional dimension that differs from at least one other of the minor lumens and/or the major lumen.

The multi-lumen catheter 600 may have different configurations of minor lumens and major lumen. In the illustrated embodiment, each of the minor lumens 608 radially surrounds the major lumen 610. In some embodiments, each minor lumen is disposed in a common plane that runs along the major lumen. Some embodiments may include minor lumens with different largest cross-sectional dimensions, e.g., to enable different medication delivery rates. In the illustrated multi-lumen catheter 600, the minor lumens 608 and the major lumen 610 have circular cross sections. However, non-circular cross sections are contemplated. Accordingly, some embodiments may have one or more minor lumens and/or a major lumen with a non-circular cross section. Some embodiments may include greater or fewer minor lumens than in the multi-lumen catheter 600 of FIG. 6A and FIG. 6B. Some embodiments may include more than one major lumen.

In some embodiments, the multi-lumen catheter 600 is formed substantially from a medical grade polymer such as [insert representative material]. In some embodiments, the multi-lumen catheter 600 is disposable and designed for single-patient use. Thus, in some methods of use, the multi-lumen catheter 600 is disposed of after use in connection with a single patient. In some embodiments, the multi-lumen catheter includes one or more in-line check valves to prevent medication backflow. In some embodiments, the multi-lumen catheter 600 is part of a kit of disposable components along with a manifold and/or an adaptor.

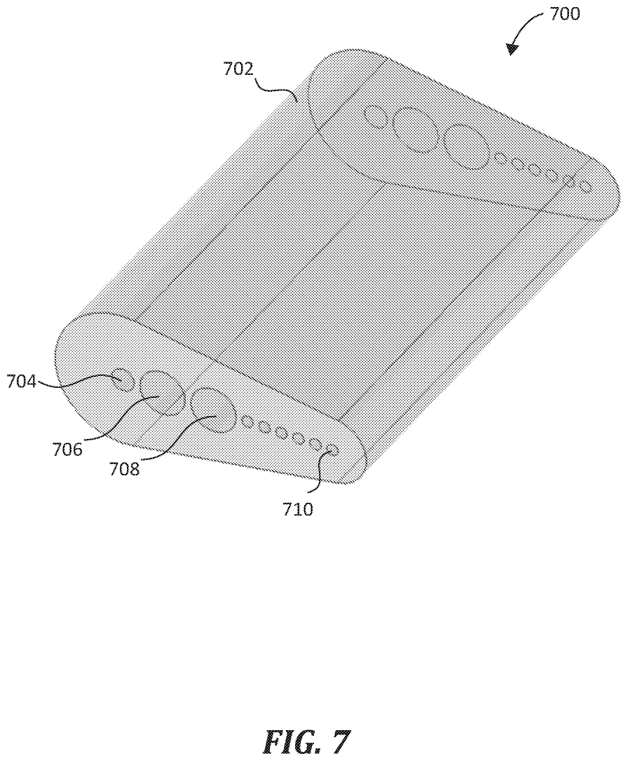

FIG. 7 shows a section of a non-limiting multi-lumen catheter 700, in accordance with another embodiment of the present disclosure. Except where stated, the multi-lumen catheter 700 has common features with the multi-lumen catheter 600 of FIG. 6A and FIG. 6B.

The multi-lumen catheter 700 has a tubing body 702 formed of medical grade polymer. The tubing body 702 has a teardrop cross sectional shape that is configured to facilitate coupling with a treatment administration device in the correct orientation, i.e., such that each of the fluidic channels of the multi-lumen catheter 700 aligns properly with the corresponding fluidic channels of the treatment administration device.

The tubing body 702 includes a first minor lumen 704, a first major lumen 706, a second major lumen 708, and a plurality of second minor lumens 710. Each of the foregoing lumens resides in a common plane, and each lumen runs substantially parallel to the other lumens along the length of the multi-lumen catheter 700. The first minor lumen 704 may be configured to open into the first major lumen 706 at a location adjacent to a downstream end. The second minor lumens 710 may be configured to open into the second major lumen 708, e.g., at a location adjacent to the downstream end. The first minor lumen 704 has a larger cross-sectional dimension than any of the second minor lumens 710. The first major lumen 706 and the second major lumen 708 have approximately equal cross-sectional dimensions.

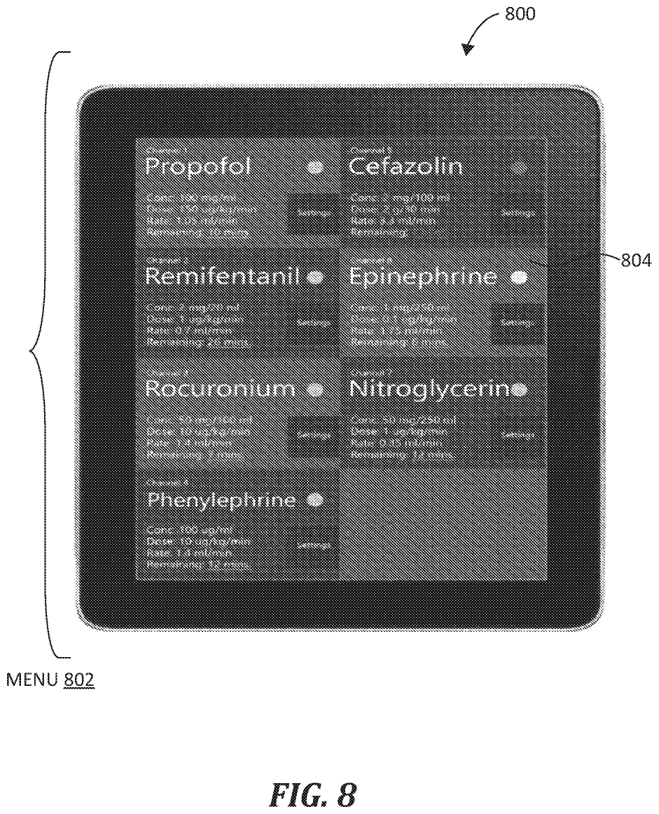

FIG. 8 illustrates a representative user interface 800, in accordance with an embodiment of the treatment selection module. The user interface 800 forms part of the treatment delivery apparatus in some embodiments. In some embodiments, the user interface 800 may be integrated into the treatment administration device of the treatment delivery apparatus. For example, the user interface 800 may be disposed at least partially within a housing of the treatment administration device so that it is readily visible and accessible. In other embodiments, the user interface 800 may be disposed on a mobile device, such as a smartphone, a tablet, or a laptop. In other embodiments, the user interface 800 may be disposed on a remote station (e.g., a desktop computer) that is remote from the treatment administration device.

The user interface 800 is configured to receive a treatment selection. Accordingly, the user interface 800 may be configured to receive inputs via a touchscreen, a keypad, a keyboard, a mouse, or other input device. The user interface 800 includes such input device(s). The user interface 800 is communicatively connected with the controller of the treatment delivery apparatus, such that the treatment selection can be communicated to the controller.

The user interface 800 is configured to present at least one menu 802 that includes a plurality of menu items 804. The user interface 800 may include one or more different menus for each functional module embodied in the controller of the treatment delivery apparatus. Representative functional modules are described below with respect to FIG. 9. Collectively, the one or more menus presented by the user interface 800 enable a user to input a treatment selection.

In the illustrated embodiment, the menu items 804 include a plurality of a medication windows. Each medication window enables selection and adjustment of one or more medications. For example, in FIG. 8, each menu item presents a medication name, a medication channel corresponding to a fluidic channel of the treatment delivery apparatus, a medication concentration, a dosage, a delivery rate, a remaining medication quantity, a settings prompt, and a visual indicator that corresponds with one or more pre-set conditions (e.g., the visual indicator turns green when the particular mediation is being administered).

The number, content, and arrangement of each of the menu items 804 is representative. In other embodiments, the user interface may present greater or fewer menu items, and the content of each menu item may differ. For example, in some embodiments, the menu 802 may be a mode menu that enables selection of a treatment mode. The mode menu is described below with respect to FIG. 9.

FIG. 9 shows a schematic representation of a treatment delivery apparatus 900 that is configured to deliver a treatment to a patient 902, in accordance with one non-limiting embodiment of the present disclosure. Unless stated otherwise, elements of the treatment delivery apparatus 900 having alike names as elements of other embodiments described herein have similar features as those elements, and vice versa.

The treatment delivery apparatus 900 includes a treatment administration device 904, a multi-lumen catheter 906 configured to fluidically connect to the treatment administration device 904, a controller 908 configured to communicatively connect to the treatment administration device 904. The treatment administration device 904 is configured to reversibly and fluidically couple with a plurality of medication cartridges 910. In some embodiments, the treatment delivery apparatus 900 includes the medication cartridges 910

The treatment administration device 904 includes a user interface 912, a plurality of cartridge-pump interfaces 914, a plurality of pumps 916, and a catheter adaptor 918. Each of the foregoing elements is at least partially disposed within a housing 920 of the treatment administration device 904. In some embodiments, the user interface 912 is not disposed at least partially within the housing 920 (e.g., it may be disposed on a remote device, such as a tablet). In some embodiments, the user interface 912 is at least partially within the housing 920, and a second user interface is disposed on a remote device.

The multi-lumen catheter 906 is configured to fluidically couple to the treatment administration device 904 via the catheter adaptor 918. Likewise, multi-lumen catheter 906 is configured for fluidic connection at a downstream end thereof with a patient's IV (i.e., a hypodermic needle), with a reservoir, and/or with one or more hardware elements (e.g., a connector, a flow regulator, a Luer lock, etc.) located upstream of the patient's IV.

The controller 908 includes a processor 922 (e.g., a general processing unit, graphical processing unit, or application specific integrated circuit); a data store 924 (a tangible machine-readable storage medium); and a plurality of modules that may be implemented as software logic (e.g., executable software code), firmware logic, hardware logic, or various combinations thereof. The controller 908 includes a communications interface having circuits configured to enable communication with the treatment administration device 904, including the user interface 912, the cartridge-pump interfaces 914, the pumps 916, the catheter adaptor 918, an electronic medical record 926, a master drug list 938, a remote server, a base station, or other network element via the internet, cellular network, RF network, Personal Area Network (PAN), Local Area Network, Wide Area Network, or other network. Accordingly, the communications interface may be configured to communicate using wireless protocols (e.g., WIFI®, WIMAX®, BLUETOOTH®, ZIGBEE®, Cellular, Infrared, Nearfield, etc.) and/or wired protocols (Universal Serial Bus or other serial communications such as RS-234, RJ-45, etc., parallel communications bus, etc.). In some embodiments, the communications interface includes circuitry configured to initiate a discovery protocol that allows the controller 908 and other network element (e.g., at least one cartridge-pump interface of the cartridge-pump interfaces 914) to identify each other and exchange control information. In an embodiment, the communications interface has circuitry configured to a discovery protocol and to negotiate one or more pre-shared keys.

As used herein, the data store 924 is a tangible machine-readable storage medium that includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

As stated above, the controller 908 includes a plurality of modules. Each module includes logic that, when executed by the processor 922, causes the treatment administration device 904 to perform one or more operations related to the safe and efficient delivery of a treatment to the patient 902. The data store 924 includes a treatment selection module 928, an inventory module 930, a treatment administration module 932, a patient information module 934, a treatment safety module 936, a treatment accountability module 940, a treatment mode module 942, and a target controlled infusion module 944. Other embodiments may include fewer or additional modules. Any of the foregoing modules may communicate with one or more of the other modules.

The treatment selection module 928 includes logic that, when executed, causes the treatment delivery apparatus 900 to present a selection of medications on the user interface 912, and to receive a treatment selection on the user interface 912. As used herein, the treatment includes at least one available medication of a plurality of available medications, such as is determined by the inventory module 930. In some embodiments, the treatment selection module 928 causes the user interface 912 to present one or more menus, each menu presenting one or more menu items (e.g., a plurality of a medication windows). In an embodiment, the treatment selection module 928 causes the user interface 912 to present an application (e.g., a search prompt or direct entry field) that enables the user to directly enter information (e.g., a medication name). In an embodiment, the treatment selection module 928 limits the menu and/or search prompt results to medications that are available to the treatment administration device 904 based upon one or more medication cartridges that are coupled with the cartridge-pump interfaces 914 (as described below with respect to the inventory module 930). In an embodiment, the treatment selection module 928 determines the available medications from signals provided by the cartridge-pump interfaces 914, one or more of which is configured to read digitally identifiable information from a medication cartridge coupled thereto. In an embodiment, the treatment selection module 928 determines the menu of medications, search results, and/or direct entry results based upon patient information that is entered via the user interface 912 and/or retrieved from the electronic medical record 926. In an embodiment, the treatment selection module 928 determines the menu items presented (e.g., which medications), search results, and/or direct entry results based upon a mode selection that is retrieved from the mode treatment selection module 928, which is described below. In some embodiments, the treatment selection module causes the user interface to display include additional prompts, fields, and/or options from those described above.

The treatment selection module 928 is configured to receive, via the menu(s), search prompt(s), and/or direct entry field(s) on the user interface 912, a treatment selection (i.e., a selection of one or more medications or other substances) for administration to the patient 902. The treatment selection is provided as inputs into the user interface 912. In some embodiments, the treatment selection includes at least one medication that is available to the treatment administration device 904 based upon one or more medication cartridges that are coupled with the cartridge-pump interfaces 914. In some embodiments, the treatment includes at least one medication that is not available from the plurality of available medications; in such embodiments, the treatment selection module 928 may prompt the user via the user interface 912 to provide the unavailable medication, e.g., by coupling an appropriate medication cartridge to one of the cartridge-pump interfaces 914.

In some embodiments, the treatment selection module 928 causes the user interface 912 to present a delivery rate, a concentration, a dose, an administered amount, a remaining amount, and/or another value for each medication that is pertinent to the treatment selection. Each of the foregoing variables may be selected and adjusted independently by the user, and may be at least partially controlled by one or more other modules described herein (such as the treatment safety module 936). FIG. 8, described above, illustrates a representative menu that may be presented on the user interface 912 as part of the treatment selection module 928. FIG. 8 also illustrates a representative treatment selection.

In some embodiments, the treatment selection module 928 is configured to communicate with other modules of the controller 908, including the inventory module 930, the patient information module 934, the treatment safety module 936, the treatment accountability module 940, the treatment mode module 942, and/or the target controlled infusion module 944. In some embodiments, communication(s) between the treatment selection module 928 and the other module(s) prevent a user from making a particular treatment selection, cause the treatment selection module 928 to suggest one or more treatment selections, and/or otherwise influence the treatment selection in ways that will become clear when each of the modules is described below.

The inventory module 930 includes logic that, when executed, causes the treatment delivery apparatus 900 to determine an “inventory” or “pharmacy” of medications that are available to the treatment administration device 904 based upon one or more medication cartridges that are coupled with the cartridge-pump interfaces 914. The inventory module 930 may determine the inventory of available medications at least partially based upon signals received from the cartridge-pump interfaces 914. In some embodiments, at least some of the cartridge-pump interfaces 914 are configured to recognize digitally identifiable information from medication cartridges coupled thereto. In such embodiments, the inventory module 930 is configured to determine the inventory of available medications based upon the digitally identifiable information. In some embodiments, the digitally identifiable information includes at least one of a medication type, medication amount, a medication concentration, a medication cartridge “type,” a time remaining to cartridge change, or an expiration date. From this digitally identifiable information, the inventory module 930 determines what medication(s) are available for provision to the multi-lumen catheter 906. The inventory module 930 is configured to provide information related to the inventory of available medications to other modules of the controller 908, such as the treatment administration module 932.

The treatment administration module 932 includes logic that, when executed, causes the treatment delivery apparatus 900 to administer one or more treatments to the multi-lumen catheter 906 based at least upon the treatment selection received from the treatment selection module 928. To administer the selected treatment, the treatment administration module 932 individually controls one or more of the pumps 916 in order to pump precise dosage(s) of one or more available medications to the multi-lumen catheter 906. The medications and dosages thereof are determined by the treatment selection. In some embodiments, the treatment administration module 932 is configured to communicate with the inventory module 930, e.g., to indicate that one or more medications of the selected treatment are unavailable or otherwise need replacing. This may cause the treatment administration module 932 to display a prompt on the user interface 912 to replace one or more medication cartridges. In some embodiments, the treatment administration module 932 is configured to communicate with one or more of the patient information module 934, the treatment safety module 936, the treatment accountability module 940, the treatment mode module 942, and/or the treatment mode module 942. In some embodiments, the treatment administration module 932 is configured to cause the treatment administration device 904 to administer the treatment when at least one of the cartridge-pump interfaces is not coupled with any of the medication cartridges.

The patient information module 934 includes logic that, when executed, causes the treatment delivery apparatus 900 to receive patient information from the user interface 912 and/or to receive patient information from the electronic medical record 926. In some embodiments, the patient information includes at least one of an allergy, an age, a weight, an ethnicity, a medical history, a medication history, a comorbidity, an ASA status, a sex, and/or potentially other patient information. In some embodiments, the patient information module 934 causes the user interface 912 to display a menu prompting a user to provide at least some of the patient information. In some embodiments, the patient information module 934 retrieves at least some of the patient information from the electronic medical record 926. The patient information module 934 is configured to provide at least some of the received patient information to other modules of the controller 908, including the treatment selection module 928, the treatment safety module 936, the treatment accountability module 940, and the target controlled infusion module 944.

The treatment safety module 936 includes logic that, when executed, causes the treatment delivery apparatus 900 to determine a standard dosage and/or a safe dosage for at least one available medication of the selected treatment. In some embodiments, the treatment safety module 936 determines the safe and/or standard dosages based at least in part upon patient information provided by the patient information module 934. In some embodiments, the treatment safety module 936 identifies a risk based upon the patient information and the treatment selection, causes the user interface 912 to display a warning based upon the risk, and controls administration of the treatment based upon the warning. In such embodiments, the treatment safety module 936 controls administration of the treatment by preventing administration of the treatment. In an embodiment, the treatment safety module 936 prevents administration of the treatment by imposing one or more “safe dosage” limits for each available medication of the treatment (such as may be retrieved from a master drug list). These “safe dosage” limits may or may not be triggered by the formulation. In an embodiment, the treatment safety module 936 prevents administration of more than a safe dosage of one or more medications of the treatment by providing a warning on the user interface when the formulation includes more than the safe dosage (i.e., more than established by one of the “safe dosage” limits). A user may override this warning. In some embodiments, the treatment safety module 936 identifies an interaction between the patient and at least one medication, causes the user interface 912 to display a warning on the user interface based upon the interaction and the treatment selection, and controls administration of the treatment based upon the warning (e.g., displays a warning and allows a user to override the warning before administering the treatment). In some embodiments, the treatment safety module 936 references the 938 and determines the safe and/or standard dosages based at least in part upon the master drug list 938. In some embodiments, based upon the master drug list 938, the treatment safety module 936 identifies an interaction between two or more medications of the selected treatment. In the foregoing embodiments, if the treatment safety module 936 identifies an interaction, the treatment safety module 936 communicates that information to one or more other modules of the 908. In some embodiments, the master drug list 938 is stored locally on the controller 908. In some embodiments, the master drug list 938 is remote from the controller 908. In some embodiments, the treatment safety module 936 is configured to communicate with the treatment selection module 928, such that the treatment selection includes safe and/or standard dosages of the medication(s) of the selected formulation. In some embodiments, the treatment safety module 936 is configured to communicate with the treatment administration module 932, such that only safe and/or standard dosages of the medication(s) are administered to the patient 902, regardless of the treatment selection. In some embodiments, the treatment safety module 936 includes logic that, when executed, causes the treatment delivery apparatus 900 to flush at least one fluidic flow path with saline or other inert solution. In some embodiments, the flushing may occur automatically after a portion of the fluidic flow path is removed or replaced, e.g., at least one of the medication cartridges 910, a manifold, a catheter adaptor, and/or the multi-lumen catheter 906. In some embodiments, the treatment safety module 936 is configured to prevent administration of certain medication(s) (e.g., controlled substances) by enacting a logon or security function (e.g., prompting a user to provide security credentials) and/or by activating a mechanical security interface (e.g., a key). For example, in an embodiment, the treatment safety module 936 is configured to determine, based upon communication with the treatment selection module 928, that the treatment requires a controlled substance (e.g., an opiate). Such determination may include communication with the master drug list 938. Upon determining that at least one medication of the treatment is a controlled substance, the treatment safety module 936 prompts the user interface 912 to display a security interface (e.g., a login interface, a digital lock, an authentication interface, etc.) and/or to cause the treatment administration unit 904 to activate a mechanical security interface (e.g., a mechanical lock). Further, the treatment safety module 936 is configured to prevent administration of the controlled substance until a user successfully overrides or bypasses the security interface (e.g., by providing sufficient credentials) and/or the mechanical security interface.

The treatment accountability module 940 includes logic that, when executed, causes the treatment delivery apparatus 900 to determine, for each available medication of the selected treatment, an administered dosage and/or an administered time. In some embodiments, the treatment accountability module 940 records the administered dosage and the administered time on the data store 924 of the controller 908. In some embodiments, the treatment accountability module 940 transmits the administered dosage and the administered time to an external destination (e.g., a remote server).

The treatment mode module 942 includes logic that, when executed, causes the treatment delivery apparatus 900 to present on the user interface 912 a mode menu, e.g., a menu of operating modes. In some embodiments, the mode menu includes at least one of the following menu items: an emergency mode, a surgical floor mode, an anesthesia mode, a medicine floor mode, an intensive care unit (ICU) mode, a pediatric mode, and a target controlled infusion mode. The treatment mode module 942 is further configured to receive a mode selection from the mode menu. Based upon the mode selection, the user interface 912 may present a different option set for each mode and/or adjust one or more parameters of the treatment delivery apparatus 900. In some embodiments, the treatment selection module 928 presents different menu items on the user interface 912 (e.g., different menus of available medications, different dosage ranges, different administration speeds, different preset treatments, etc.), search results, and/or direct entry results based upon the mode selection received by the mode treatment selection module 928. In an embodiment, ICU mode includes additional alarms, has a simplified user interface, and/or disables administration of dosages of one or more available medications other than a prescribed dosage. In an embodiment, the emergency mode and/or the surgical floor mode include alarms that are more easily silenced as compared to other modes. In an embodiment, the emergency mode and/or the surgical floor mode has a visual-alarm feature.

The target controlled infusion module 944 includes logic that, when executed, causes the treatment delivery apparatus 900 to formulate a treatment based upon a target controlled infusion input set, which may be input via the user interface 912. The target controlled infusion input set includes at least one of: a target anesthetic state, a target pain state, a target memory state, and/or a target hemodynamic state. In some embodiments, the treatment selection module 928 presents a menu that includes one or more menu items corresponding to the foregoing variables. The treatment administration module 932 is configured to administer the treatment formulated by the target controlled infusion module 944. In some embodiments, the target controlled infusion module 944 communicates with other modules, e.g., the patient information module 934 and the treatment safety module 936. In some embodiments, the target controlled infusion module 944 communicates with the electronic medical record 926 and/or the master drug list 938 to formulate the treatment based upon the target controlled infusion input set. In some embodiments, the target controlled infusion module 944 communicates with the treatment administration module 932 so that the treatment is administered based upon the target controlled infusion input set.

The foregoing modules are representative and not limiting. Some embodiments of the treatment delivery apparatus include additional, fewer, or different modules from those described above.

Generally, connections between operative components of the treatment delivery apparatus may be wired or wireless, and may be direct or indirect. Regardless, any component of the treatment delivery apparatus 900 can be connected to a network that ultimately provides a connection to any other component.

Treatment delivery apparatuses disclosed herein utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

In an embodiment, circuitry includes one or more ASICs having a plurality of predefined logic components. In an embodiment, circuitry includes one or more FPGA having a plurality of programmable logic components. In an embodiment, circuitry includes hardware circuit implementations (e.g., implementations in analog circuitry, implementations in digital circuitry, and the like, and combinations thereof). In an embodiment, circuitry includes combinations of circuits and computer program products having software or firmware instructions stored on one or more computer readable memories that work together to cause a device to perform one or more methodologies or technologies described herein. In an embodiment, circuitry includes circuits, such as, for example, microprocessors or portions of microprocessor, that require software, firmware, and the like for operation. In an embodiment, circuitry includes an implementation comprising one or more processors or portions thereof and accompanying software, firmware, hardware, and the like. In an embodiment, circuitry includes a baseband integrated circuit or applications processor integrated circuit or a similar integrated circuit in a server, a cellular network device, other network device, or other computing device. In an embodiment, circuitry includes one or more remotely located components. In an embodiment, remotely located components are operatively connected via wireless communication. In an embodiment, remotely located components are operatively connected via one or more receivers, transmitters, transceivers, or the like.

In an embodiment, the treatment delivery apparatus includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, circuitry includes one or more user input/output components that are operatively connected to at least one computing device to control (electrical, electromechanical, software-implemented, firmware-implemented, or other control, or combinations thereof) administration of a treatment.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

1. A treatment delivery apparatus, comprising:
a treatment administration device having:
a housing;
a plurality of cartridge-pump interfaces disposed within the housing; and
a plurality of pumps disposed within the housing, each pump being in fluidic connection with at least one cartridge-pump interface of the plurality of cartridge-pump interfaces;
a multi-lumen catheter reversibly couplable with the treatment administration device; and
a controller operatively connected to the treatment administration device, wherein the controller comprises a processor and logic which, when executed by the processor, causes the treatment administration device to perform operations including:
receiving a treatment selection via a user interface operably connected with the controller;
determining a plurality of available medications based upon a plurality of medication cartridges coupled with the plurality of cartridge-pump interfaces; and
administering a treatment to the multi-lumen catheter, based at least upon the treatment selection, wherein the treatment comprises at least one available medication of the plurality of available medications, wherein for the at least one available medication of the treatment, administering the treatment comprises controlling a flow from at least one medication cartridge of the plurality of medication cartridges to the multi-lumen catheter.

2. The treatment delivery apparatus of claim 1, wherein the treatment administration device comprises a catheter adaptor in fluidic connection with the plurality of pumps, wherein a plurality of fluidic flow paths extend through the catheter adaptor.

3. The treatment delivery apparatus of claim 2, wherein the catheter adaptor has an upstream portion with a first cross sectional area, and a downstream portion with a second, smaller, cross sectional area, and wherein each of the plurality of fluidic flow paths extends through both the first cross sectional area and the second cross sectional area.

4. (canceled)

5. The treatment delivery apparatus of claim 1, wherein administering the treatment is based on patient information received by the controller.

6. The treatment delivery apparatus of claim 5, wherein the patient information is received from an electronic medical record.

7. The treatment delivery apparatus of claim 5, wherein the patient information comprises at least one of an allergy, an age, a weight, an ethnicity, a medical history, a medication history, a comorbidity, an ASA status, or a sex.

8. The treatment delivery apparatus of claim 5, wherein the controller comprises logic which, when executed by the processor, causes the treatment delivery apparatus to perform additional operations, including: identifying a risk based upon the patient information and the treatment selection, displaying a warning on the user interface based upon the risk, and controlling administration of the treatment based upon the warning.

9. The treatment delivery apparatus of claim 1, wherein the controller comprises logic which, when executed by the processor, causes the treatment delivery apparatus to perform additional operations, including: identifying an interaction between at least two of the plurality of available medications, displaying a warning on the user interface based upon the interaction and the treatment selection, and controlling administration of the treatment based upon the warning.

10. The treatment delivery apparatus of claim 1, wherein the controller comprises logic which, when executed by the processor, causes the treatment delivery apparatus to perform additional operations, including: determining a safe dosage for each available medication of the treatment,
wherein administering the treatment comprises preventing administration of more than the safe dosage for each available medication of the treatment.

11. The treatment delivery apparatus of claim 10, wherein the controller determines the safe dosage by referencing a master drug list.

12. The treatment delivery apparatus of claim 1,
wherein administering the treatment comprises, for each available medication of the treatment, determining an administered dosage and an administered time, and
wherein the controller comprises logic which, when executed by the processor, causes the treatment delivery apparatus to record the administered dosage and the administered time on a memory of the controller.

13. The treatment delivery apparatus of claim 1, wherein the controller comprises logic which, when executed by the processor, causes the treatment delivery apparatus to perform additional operations, including:
presenting a mode menu having at least one of an emergency mode, a surgical floor mode, an anesthesia mode, a medicine floor mode, an intensive care unit (ICU) mode, a pediatric mode, or a target controlled infusion mode;
receiving a mode selection from the mode menu; and
presenting an option set on the user interface based upon the mode selection.

14. The treatment delivery apparatus of claim 1,
wherein receiving the treatment selection comprises receiving a target controlled infusion input set that comprises at least one of a target anesthetic state, a target pain state, a target memory state, or a target hemodynamic state,
wherein administering the treatment is based upon the target controlled infusion input set.

15. The treatment delivery apparatus of claim 1,
wherein the multi-lumen catheter comprises a plurality of minor lumens running along a major lumen of the multi-lumen catheter,
wherein administering the treatment comprises, for each available medication of the treatment, controlling the flow of the available medication from at least one medication cartridge of the plurality of medication cartridges to at least one minor lumen of the multi-lumen catheter.

16. The treatment delivery apparatus of claim 15, wherein when the multi-lumen catheter is reversibly coupled with the treatment administration device, each minor lumen of the multi-lumen catheter is fluidically coupled with at least one pump.

17. The treatment delivery apparatus of claim 15, wherein each minor lumen opens into the major lumen at a location adjacent to a downstream end of the multi-lumen catheter.

18. (canceled)

19. The treatment delivery apparatus of claim 15, wherein each minor lumen radially surrounds the major lumen.

20. The treatment delivery apparatus of claim 15, wherein each minor lumen is disposed in a common plane that runs along the major lumen.

21. (canceled)

22. The treatment delivery apparatus of claim 15, wherein the major lumen and each minor lumen has a circular cross-sectional shape.

23. The treatment delivery apparatus of claim 15, wherein the multi-lumen catheter comprises an emergency medication port located proximal to a downstream end of the multi-lumen catheter and providing access to the major lumen.

24.-31. (canceled)
